(19) United States
(12) Patent Application Publication
(10) Pub. No.: US 2007/0092578 A1
(43) Pub. Date: Apr. 26, 2007

(54) Skin rejuvinating supplement

(76) Inventors: Yasuhiro Itagaki, New York, NY (US); Nobuko Kanayama, New York, NY (US); Yasuhisa Iida, Astoria, NY (US); Jun Nakanishi, Cliffside Park, NJ (US)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 11/258,182

(22) Filed: Oct. 26, 2005

(51) Int. Cl. A61K 36/45; A61K 35/56; A61K 31/59; A61K 31/12

(52) U.S. Cl. 424/547; 514/167; 514/690; 424/732

(57) ABSTRACT

A skin rejuvenating supplement composition for treating age spots, darkening of the skin, dry skin, calcium amino acid deficiencies, osteoporosis and eye site deficiencies. The composition contains 100 to 1,500 mg of pearl powder, 0.6 to 50 μkg Vitamin D, 1.5 to 20.0 mg of astaxanthin and 20 to 750 mg of bilberry extract. The composition can be administered in a tablet, also containing glycerol in an amount of about 500 mg, once or two times per day.

### BACKGROUND OF THE INVENTION

The skin is the body's outer covering and the body's largest organ. Skin protects the body against heat, light, injury, and infection. It regulates body temperature and stores water, fat, and vitamin D. Damage to the skin from normal environmental exposures has been well documented. The skin is considered a highly stressed organ. It is subject to chemical attack and also to constant exposure to ultraviolet radiation. Prolonged exposure to ultraviolet light is linked to skin damage characterized by sunburn, aging and cancer. The production of free radicals from oxygen in the presence of ultraviolet light is linked to degradation of collagen and other structural components of the skin in addition to DNA mutation and structural changes which impair the skin's ability to heal itself. In recent years the demand for cosmetic compositions and cosmetic methods for improving the appearance and condition of skin has grown enormously.

Consumers are increasingly seeking “anti-ageing” skin care products that treat or delay the visible signs of chronoageing and photoageing skin, such as wrinkles, lines, sagging, hyperpigmentation and age spots.

Consumers also frequently seek other benefits from skin care products in addition to anti-ageing. The concept of “sensitive skin” has also raised the consumer demand for skin care products that improve the appearance and condition of sensitive, dry and/or flaky skin and to soothe red, and/or irritated skin.

In China, pearl powder has been known for thousands of years as traditional medicine. It is used to treat cataract and ringing in the ear, bronchitis, high blood pressure, inflammation of the liver (hepatitis), angina, autonomic imbalance, etc. It is also used in its raw form as a skin whitener/rejuvenator, anti-aging agent, as well as an anti-inflammatory agent.

Conchiolin is a protein extracted from pearl powder. There are several patents based on conchiolin, or its hydrolyzate, made from shells and pearls of pearl oysters, sea mussels, etc. Patent JP-H06-17123 recognizes conchiolin-vitamin E compound to be a potent antioxidant. Patent JP-H06-211640 recognizes conchiolin as an active oxygen suppressor. Patent JP 2003-292447 recognizes pearl powder compound as anti-diabetes supplement.

Other patent literature includes an astaxanthin-bilberry extract compound which has been reported in patent JP-2002-17295 showing effectiveness against eye diseases while helping to maintain optical functions. Patent JP-2002-335668 recognizes astaxanthin-tocotrienol compound as a skin-beautifying agent. Patent JP-2004-210725 recognizes astaxanthin to have cyclooxygenase inhibitory function.

### DETAILED DESCRIPTION OF THE INVENTION

The present invention is a formulation including at least three ingredients: conchiolin protein, vitamin D and an antioxidant. The formulation of the present invention is intended for human health care, in particular skin care. Specifically, it may be used for treating age spots, darkening of the skin (pigmentary deposits) and dry skin. It may also be used for treating calcium/amino acid deficiencies, osteoporosis and for improving eyesight. In fact, the present invention is the first invention that rejuvenates the skin and at the same time prevents osteoporosis.

Conchiolin protein is the protein that gives pearl the rainbow-like or iridescent glow. Conchiolin protein can be found in pearls produced by bivalve shellfishes such as pearl oysters, abalone, silver-lipped pearl oysters (pinctada maxima), black-lipped pearl oysters (pinctada margaritifera), etc. It is woven into the pearl's calcium. To extract the protein from a pearl, which is primarily calcium, the pearl needs to be dissolved first with acid that reacts with calcium carbonate such as hydrochloric acid. However, for use with this invention, conchiolin protein or its hydrolysate need not be extracted and purified. Rather, it can be used in a form (powder, liquid etc.) that can be absorbed readily by the body via internal organs such as the stomach and intestines. One such form is pearl powder. Pearls that are ground into powder provide an adequate source of conchiolin protein or its hydrolysate. Pearl powder can be made fine so that the powder particles have an average diameter of 1 to 10 μm.

Vitamin D is an agent good for aiding the digestion function of the human body. It assists the body in digesting not only Vitamin D itself but other substances as well. For example, in combination with pearl powder, Vitamin D aids the body in digesting and absorbing conchiolin protein and calcium. Used in the solid form, Vitamin D creates an easily absorbed effective agent for helping the body digest and absorb surrounding ingredients.

Antioxidant is a classification of substances which are inhibitors, effective in preventing or slowing down oxidation by singlet oxygen and/or various forms of radicals. Anitoxidants include vitamins C and E, vitamin A (which is converted from beta-carotene), selenium (a mineral), lycopene, and a group known as the carotenoids. One antioxidant for the present invention is astaxanthin, which is the main carotenoid pigment found in aquatic animals and algae. Additional antioxidants that can be used for the present invention include carnosine, cartenoid, co-enzyme Q10, Vitamins A, B, C and E, selenium, soy isoflavones, and zinc. Astaxanthin is used because of its properties as a strong antioxidant and an effective compound against UV radiation on the skin.

The formulation of the present invention is generally administered orally. It may be administered in an individual dosage form such as capsule form, in which case there will be additional ingredients, generally considered to be inactive ingredients, such as fillers. Glycerol may be used as such an inactive ingredient.

Generally one dosage unit of the present invention will have the following amounts of compounds: the amount of conchiolin found in 100 to 1,500 mg of pearl powder, 0.6 to 50 μg of Vitamin D, and an amount of an antioxidant, such as 1.5 to 20.0 mg of a strong antioxidant like astaxanthin or several more times (2 to 10 times more) of a weaker antioxidant.

Within the total ranges of amounts of each ingredient, smaller amounts are possible, for example, the amount of conchiolin found in 200, 400, 600, 900, 1,000 or 1,200 mg of pearl powder, 2.5, 5, 10, 20, 30, or 40 μg of Vitamin D and 2, 4, 6, 8, 10, 12, 15, 17 or 18 mg of a strong antioxidant like astaxanthin. Combinations of these amounts may be used to create a dosage unit for administering the present invention.

Preferably, when in an individual dosage form, the content of a typical daily dosage will be 100 to 300 mg of pearl powder, 0.6 to 1.25 μg of Vitamin D and 1.5 to 10.0 mg of astaxanthin, with Vitamin D not exceeding 100 IU. Glycerol, or other inactive ingredients, can be combined with the above active ingredients in a variable amount to formulate a capsule or tablet for taking orally. Typically, the formulation will be designed for administration twice a day, so the amounts of components in a dosage unit would be 50 to 150 mg of pearl powder; 0.3 to 0.63 μg of Vitamin D and 0.75 to 2.0 mg of astaxanthin.

The upper limit of the general dosage amount of each ingredient represents an amount, assuming the present invention is taken twice a day (in other words a dosage of 2×), that will not exceed the US Food and Drug Administration recommended daily allowance for an adult human.

The lower limit of the effective dosage amount of each ingredient represents the lower limit of half of the daily amount necessary to achieve the objective of the present invention.

Polyphenols, a class of phytochemicals found in high concentrations in wine, tea, grapes and a wide variety of other plants, may also be added to the above formulation to further improve the present invention, as polyphenols are good protectors against UV radiation on the skin. Polyphenols can also act as antioxidants. Bilberry extract may be used as an ingredient because it is an excellent source of polyphenols in addition to being an antioxidant. In fact, by using astaxanthin and bilberry extract, both of which are a 100˜1,000 times more effective antioxidant as compared to vitamin E, the age-spot prevention/skin restoration functions, at least one objective of this invention, has been dramatically improved.

Thus, when in an individual dosage form, the content of a typical dosage unit of the present invention will be 100 to 300 mg of pearl powder, 0.6 to 1.25 μg of Vitamin D, 1.5 to 4.0 mg of astaxanthin, and 20 to 750 mg of bilberry extract, with Vitamin D not exceeding 100 IU/day. Within the range of amount of bilberry extract, smaller amounts are possible like 150, 300, 450, 500, and 650 mg. However for a typical daily dosage 150 mg of bilberry extract will be sufficient. Glycerol can be combined with the above ingredients in a variable amount to formulate a capsule or tablet for taking orally. Typically, the formulation will be designed for administration twice a day, so the amount of Vitamin D will typically not exceed 50 IU per dosage unit.

### EXAMPLE

An example will be given of an embodiment of the present invention. For this example, pearl powder in powder form (super-fine Nano Tec Pearl Powder with an average particle diameter size of 1 μm) was purchased from Lucoral Inc. and astaxanthin in solid form, Vitamin D in solid form, bilberry extract in solid form, and glycerol were purchased from Sigma-Aldrich, Inc.

The ingredients were then combined as follows: 250 mg super-fine pearl powder; 3.3 mg astaxanthin; 100 mg bilberry extract; 1.25 μg of Vitamin D and 500 mg glycerol were placed in a glass flask and homogenized with a magnetic stirrer. The temperature was kept at about 50° C. using a hot water bath. The ingredients were mixed until a thick syrup-like liquid was produced. This liquid was poured into a capsule for oral consumption.

To prove the effects of the present invention, the above formulated capsules were taken by 10 subjects (Sub), male (M) and female (F) (Sub 1 to Sub 10; M and F below) ranging from 30's to 50's in age, for a period of two months. Each subject took approximately two capsules a day. The results are summarized below in the table.

In the clinical trials, nine categories (categ.) were measured:
White=feels white/more transparent
Finer=feels finer
AS=age spots vanished/fading
Firm=feels firmer
Moist=feels more moist
Mk-up=makeup spreads better
LessC=needs less concealer
Body=entire body feels whiter

Eyes=eyes feel less strained

TABLE
The following is a summary of the clinical trials:

| Age/ | Sub 1 | Sub 2 | Sub 3 | Sub 4 | Sub 5 | Sub 6 | Sub? | Sub 8 | | |
|---|---|---|---|---|---|---|---|---|---|---|
| categ. | 40 s F | 30 s F | 30 s F | 50 sM | 30 s F | 50 s F | 40 s F | 50 sM | Sub 950 sM | Sub 1040 sM |
| White | •• | •• | •• | • | | • | | • | •• | • |
| Finer | • | •• | • | • | • | | | • | •• | |
| AS | | •• | •• | •• | • | • | | • | •• | •• |
| Firm | | •• | • | •• | | | | • | | |
| Moist | | •• | •• | • | | | | • | | • |
| Mk-up | • | •• | • | | | | | | | |
| LessC | | | •• | | | | | | | |
| Body | | | • | • | | • | | • | | •• |
| Eyes | | • | | • | | | • | • | | |

•• = very much
• = somewhat

Several visual observations were made about the clinical trials. First, age spots on older subjects, male and female, became smaller in size (the area the covered on the face) and they also became lighter or more white. Second, other types of spots on the skin, perhaps due to sun damage or other skin problems, became smaller, whiter and some completely disappeared. Thus the total number of spots on the skin of the face became fewer. When spots on the face disappeared, the skin color returned to a smoother, consistent color from a more motley color.

In the clinical trials, it took one month to two months for the results to appear, which coincides with the normal skin-regeneration cycle of 28 days to 60 days. A person should take the invention daily as long as the person wishes to benefit from its effectiveness. Dosage interruption or stoppage may cause undesired conditions, such as age spots and dry skin, to reappear.

We have now found that effective treatment and prevention of normal, but cosmetically undesirable, skin conditions, due to chronoageing or photoageing, such as wrinkles, lines, sagging, hyperpigmentation and age spots, may be obtained through the use of the present invention for the skin. We have also found that the use of such cosmetic compositions advantageously provides further skin care benefits in addition to anti-ageing such as soothing sensitive and/or irritated skin, controlling oil/sebum secretion and for lightening the skin.

In addition the present invention can be used to prevent osteoporosis. If dosage units of the present invention are taken regularly, for example once or twice a day for 3 months, 6 months, 1 year, 2 years, 5 years or more, the invention can effectively deliver calcium to the body, which will help prevent osteoporosis.

While there have been illustrated and described several embodiments of the present invention, it will be apparent that various changes and modifications thereof will occur to those skilled in the art. It is intended in the appended claims to cover all such changes and modifications that fall within the true spirit and scope of the present invention.

What is claimed is:

1. An oral supplement composition for maintaining and improving skin condition comprising the following ingredients:
a) conchiolin protein
b) Vitamin D, and
c) an antioxidant.

2. The composition of claim 1, wherein the antioxidant is astaxanthin.

3. The composition of claim 1, wherein the conchiolin protein is provided in the composition in the form of pearl powder which incorporates the conchiolin protein.

4. The composition of claim 1 further comprising a polyphenol.

5. The composition of claim 1 further comprising a polyphenol different from the antioxidant.

6. The composition of claim 5, comprising bilberry extract as the source of the polyphenol.

7. An oral supplement composition for maintaining and improving skin condition comprising:
a) 100 to 1,500 mg of pearl powder as the conchiolin protein,
b) 0.6 to 50 μg Vitamin D, and
c) 1.5 to 20.0 mg of astaxanthin as the antioxidant.

8. The composition of claim 7 further comprising 20 to 750 mg of bilberry extract.

9. An oral supplement dosage unit for maintaining and improving skin condition comprising:
a) 100 to 300 mg of pearl powder,
b) 0.6 to 1.25 μg Vitamin D, and
c) 1.5 to 10.0 mg of astaxanthin.

10. The dosage unit of claim 9, having 250 mg of pearl powder, 1.25 μg Vitamin D and 3.3 mg of astaxanthin.

11. The dosage unit of claim 9, wherein the ingredients are contained in a capsule with 500 mg of glycerol.

12. The dosage unit of claim 9, wherein the pearl powder has an average particle diameter size of 1 μm.

13. The dosage unit of claim 9, further comprising 20 to 150 mg of bilberry extract.

14. The dosage unit of claim 13 for maintaining and improving skin condition having:
a) 250 mg of pearl powder,
b) 1.25 μg Vitamin D,
c) 3.3 mg of astaxanthin, and
d) 100 mg bilberry extract.

15. The dosage unit of claim 14, wherein the ingredients are contained in a capsule with 500 mg of glycerol.

16. A method of preventing osteoporosis comprising:
taking at least one oral supplement dosage unit comprising:
a) 100 to 1,500 mg of pearl powder,
b) 0.6 to 50 μg Vitamin D, and
c) 1.5 to 20.0 mg of astaxanthin.

17. The method of claim 16, wherein the oral supplement dosage unit further comprises 20 to 750 mg of bilberry extract.
